(19) United States
(12) Patent Application Publication
(10) Pub. No.: US 2010/0217316 A1
(43) Pub. Date: Aug. 26, 2010

(54) Suture treatment method and kit therefor

(76) Inventor: James J. Fedinec, Darien, IL (US)

Correspondence Address: JAMES J. FEDINEC, MD, 8145 CHAPMAN COURT, DARIEN, IL, 60561, US

(21) Appl. No.: 12/380,222

(22) Filed: Feb. 25, 2009

(51) Int. Cl. A61B 17/04

(52) U.S. Cl. 606/228

(57) ABSTRACT

A suture method and kit enables health care providers and patients to select colored sutures from a wide variety of colors. The colored sutures enable the patient to adorn a skin wound site so as to more effectively treat the wound with patient input and cooperation. The suturing method involves the steps of assessing a suture site or skin wound; selecting a suturing technique based on the assessed suture site or skin wound; providing a visual depiction of exemplary suture layout schema to the patient, which exemplary suture layout schema are reflective of the selected suturing technique; providing a patient with a selection of suture colors; inviting the patient to select at least one suture color from the suture color palette; selecting at least one suture color from the selection of suture colors; and suturing a suture site with a colored suture as selected by the patent.

### BACKGROUND OF THE INVENTION

1. Field of the Invention

The present invention generally relates to a method for treating skin wound sites via sutures. More particularly, the present invention relates to a suturing method and/or suture kit for outfitting or adorning a skin wound site by way of sutures of varying colors and patterns.

2. Description of the Prior Art

A suture is a threadlike medical device that doctors, and especially surgeons, use to hold skin, internal organs, blood vessels and all other tissues of the human body together, after they have been severed by injury or surgery. The threadlike devices typically have relatively high tensile strength; are non-toxic, non-wicking, and hypoallergenic. Further, they typically have a high degree of flexibility and easy to work with so that suture sites may be quickly attended to, and sutures may be quickly knotted.

State of the art, non-absorbable sutures are typically formed from polyethylene or other high strength polymeric materials and come in a limited variety of colors; usually black or blue/violet. When outfitted upon an external suture site, the lack of color variety or the limited black/blue color scheme leaves patients with a visibly unattractive wound and treatment structure. The prior art that addresses colored sutures for wound treatment is limited in scope. A pair of pertinent prior art discloses are briefly described hereinafter.

For example, U.S. Pat. No. 6,994,719 ('719 Patent), which issued to Grafton, discloses a High Strength Suture with Colored Trace at One End. The '719 Patent describes a high strength abrasion resistant surgical suture material with improved tie down characteristics that is color coded for visualization and identification purposes. The suture features a multifilament cover formed of strands of ultra high molecular weight long chain polyethylene braided with polyester, nylon or a bioabsorbable material. Selected nylon fibers in the cover are provided in a color contrasting with the other cover fibers to provide an identifiable trace. The cover surrounds a core formed of twisted strands of ultrahigh molecular weight polyethylene. The suture, provided in a #2 size, has the strength of #5 Ethibond, is ideally suited for most orthopedic procedures, and can be attached to a suture anchor or a curved needle. The identifiable trace preferably is provided along one half of the length of the suture, so that when the suture is loaded onto a suture anchor, for example, the two legs of the length of suture on either side of the suture anchor can be readily identified.

U.S. Pat. No. 7,029,490 ('490 Patent), which issued to Grafton et al., discloses a High Strength Suture with Coating and Colored Trace. The '490 Patent also describes a high strength abrasion resistant surgical suture material with improved tie down characteristics that is color coded for visualization and identification purposes. The suture features a multifilament cover formed of strands of ultra high molecular weight long chain polyethylene braided with polyester, nylon or a bioabsorbable material. Selected nylon fibers in the cover are provided in a color contrasting with the other cover fibers to provide an identifiable trace. The cover surrounds a core formed of twisted strands of ultrahigh molecular weight polyethylene. The suture, provided in a #2 size, has the strength of #5 Ethibond, is ideally suited for most orthopedic procedures, and can be attached to a suture anchor or a curved needle.

The colored strands/sutures of the foregoing disclosures are intended to operate primarily for the health care provider's benefit. The prior art thus perceives a need for a suturing method whereby patients may be able to select a suture color for their particular wound as a means to improve or enhance the patient's mental or emotional reaction(s) to wound treatment and to shift focus away from the wound per se and more toward wound treatment via wound adornment as directed or aided through patient input and the availability of a wide range of suture colors. The present invention attempts to address this need in the prior art.

### SUMMARY OF THE INVENTION

The present invention thus attempts to structurally address the foregoing concerns and thus provides a method and kit for enabling patients to select colored sutures from a wide variety of colors. The colored sutures and suturing techniques together enable the patient to adorn a skin wound site as a means to more effectively treat the wound with the cooperation of the patient.

The present invention thus provides a suturing method and kit for adorning a suture site, which method essentially comprises the steps of: assessing a suture site or skin wound; selecting a suturing technique based on the assessed suture site or skin wound; and providing a visual depiction of exemplary suture layout schema to the patient, which exemplary suture layout schema are reflective of the selected suturing technique.

The patient may then be provided with a selection of suture colors or a suture color palette at or about which time the patient may be invited to select at least one suture color from the suture color palette. The patient may thus select at least one suture color from the selection of suture colors; and the suture site may be sutured with a colored suture as selected by the patent. It is contemplated that the suture kit may well comprise any number of variously colored sutures along with surgical needles to support the suturing method.

### BRIEF DESCRIPTION OF THE DRAWINGS*

*All drawing illustrations adapted from: Surgery/[edited by] Bruce E. Jarrell, R. Anthony Carabasi III., 2^{nd} ed., Harwal Publishing, 1991, FIG. 26-1.

Other features of my invention will become more evident from a consideration of the following brief description of patent drawings:

FIG. 1 is a diagrammatic depiction of a first suturing technique for closing a skin wound showing a simple interrupted suture schematic.

FIG. 2 is a diagrammatic depiction of a second suturing technique for closing a skin wound showing a vertical mattress suture schematic with a first color pattern.

FIG. 2(a) is a diagrammatic depiction of the second suturing technique for closing a skin wound showing the vertical mattress suture schematic with a second color pattern.

FIG. 2(b) is a diagrammatic depiction of the second suturing technique for closing a skin wound showing the vertical mattress suture schematic with a third color pattern.

FIG. 2(c) is a diagrammatic depiction of the second suturing technique for closing a skin wound showing the vertical mattress suture schematic with a fourth color pattern.

FIG. 3 is a diagrammatic depiction of a third suturing technique for closing a skin wound showing a continuous over-and-over suture schematic with a first color pattern.

FIG. 3(a) is a diagrammatic depiction of the third suturing technique for closing a skin wound showing the continuous, running, or over-and-over suture schematic with a second color pattern.

FIG. 4 is a diagrammatic depiction of a fourth suturing technique for closing a skin wound showing a horizontal mattress suture schematic with a first color pattern.

### DETAILED DESCRIPTION OF THE PREFERRED METHODOLOGY**

Sutures or stitches may be classified into two main groups—those that are absorbable and those that are non-absorbable. Absorbable sutures or stitches are designed to break down harmlessly in the body over time without intervention. Non-absorbable sutures or stitches must be manually removed if they are not left indefinitely. The type of suture used varies on the operation, with the major criteria being the demands of the location and environment. The attending health care provider typically assesses the location and environment of the wound site and decides upon a preferred treatment method for suture-closing the wound site.

Sutures to be placed internally would require re-opening if they were to be removed. Sutures which lie on the exterior of the body can be removed within minutes, and without re-opening the wound. As a result, absorbable sutures are typically used internally whereas non-absorbable sutures are typically used externally. Exceptions to these general rules, however, are present. For example, sutures to be placed in a stressful environment, for example the heart, the bladder, or sternum may require specialized or stronger materials to perform their role. Typically, such sutures are either specially treated, or made of special materials, and are often non-absorbable to reduce the risk of degradation.

The present methodology concerns itself primarily with externally exposed sutures or stitches, which are readily viewable by the patient and passersby. The primary objective of the present invention is to involve the patient in the treatment process whereby the patient may be able to select a suture color and/or pattern more to their liking, and thereby shift focus away from the wound itself and more toward the treatment aspects. In other words, in a manner akin to a patient making a selection of cast color(s), it is contemplated that a patient-selected colored suture scheme may well improve the patient's cognitive approach to the treatment process.

Non-absorbable sutures are typically made of materials which are not metabolized by the body, and are used therefore either on skin wound closure, where the sutures can be removed after a few weeks, or in some inner tissues in which absorbable sutures are not adequate (e.g. heart and/or bladder).

While there are several materials used for non-absorbable sutures, perhaps the most common non-absorbable sutures are made of artificial polymeric threadlike fibers such as polypropylene, polyester or nylon. For example, PROLENE sutures are polypropylene sutures that are most often provided with blue pigmentation and ETHILON sutures are nylon sutures that are most often provided with black pigmentation.

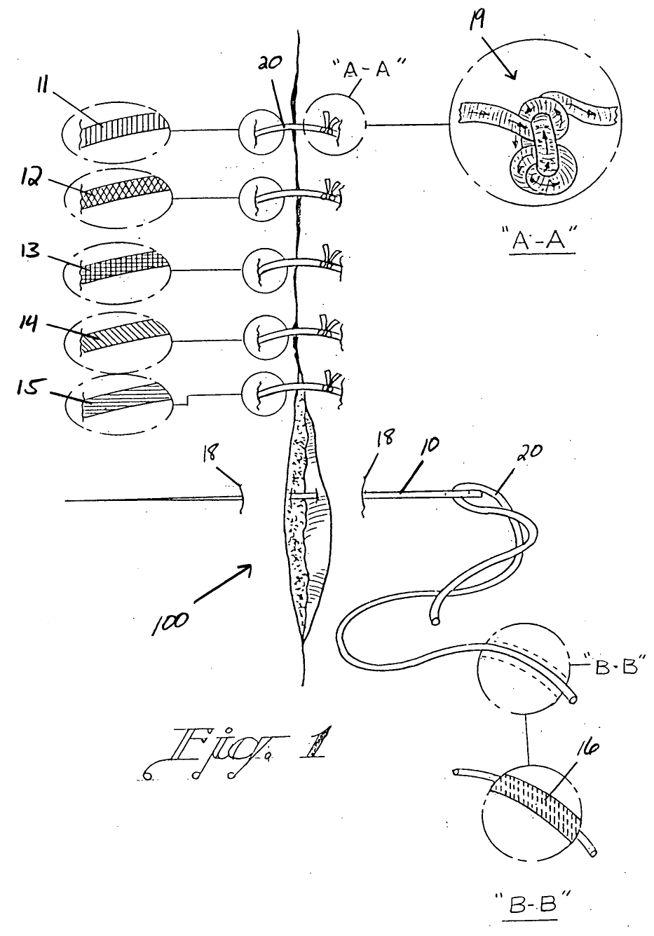

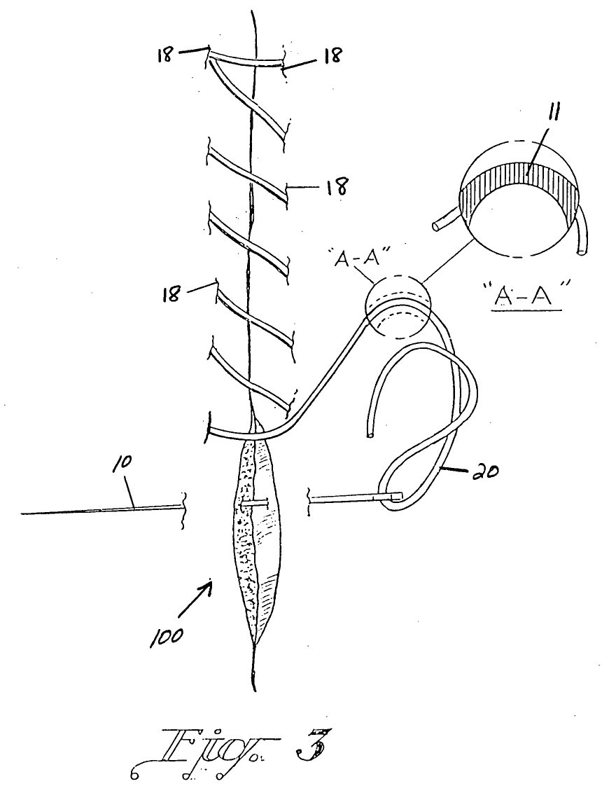

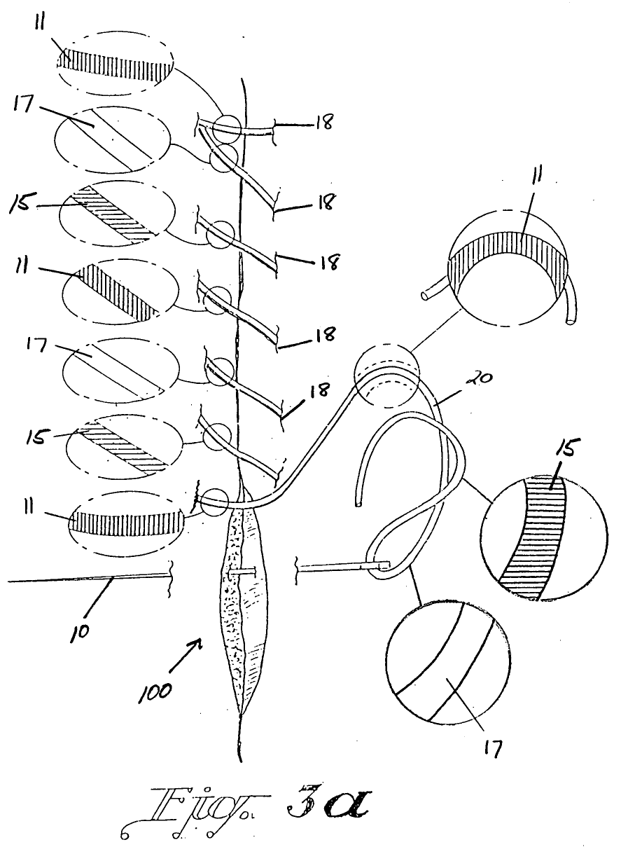

Sutures are coupled to surgical needles prior to suturing a wound site. In this regard, there are two general classifications of surgical needles, namely, traumatic needles and atraumatic needles. Traumatic needles comprise holes or eyes through which the suture must be threaded on site. A traumatic needle 10 is illustrated in FIGS. 1, 3, and 3(a). It may be seen that the suture or thread comes out of the needle's hole or eye on both sides. When passing through tissue, this type of suture rips the tissue to a certain extent, thus prompting the descriptor: “traumatic”.

Atraumatic needles with sutures comprise an eyeless needle attached to a specific length of suture thread. The suture manufacturer typically swages the suture thread to the eyeless atraumatic needle at the factory. There are several advantages to having the needle pre-mounted on the suture, including reduced suturing time (i.e. the health care provider does not have to spend time threading the suture on the needle) and the fact that with atraumatic sutures, the suture end of a swaged needle is smaller than the needle body thus minimizing tissue trauma. Nearly all modern sutures feature swaged atraumatic needles. Notably, atraumatic needles may be permanently swaged to the suture or may be designed to “pop-off” the suture with a sudden force. These so-called “pop-offs” are commonly used for interrupted sutures, where each suture is only passed once and then tied.

Suture gauges are defined by the United States Pharmacopeia (U.S.P.). Sutures were originally manufactured ranging in size from #1 to #6, with #1 being the thickest. A #4 suture might be roughly the diameter of a tennis racquet string. As manufacturing techniques improved, #0 was added to the suture diameters, and later, thinner and thinner threads were manufactured, which were identified as #00 (#2-0 or #2/0) to #000000 (#6-0 or #6/0).

Modern sutures range from #5 (i.e. a heavy braided suture typically utilized in orthopedic applications) to #11-0 (i.e. a fine monofilament suture typically utilized in ophthalmic applications). It is contemplated that the present invention incorporates the use of suture gauges having visually perceptible thicknesses or diameters so that the patient and/or passerby may be able to visually perceive the color and/or pattern scheme selected by the patient.

Referring now to the drawings with more specificity, the preferred methodology of the present invention concerns a skin wound treatment method for externally adorning a skin wound site (as generally referenced at 100) with suture(s) so as to improve the visual appearance of the wound site 100. The skin wound treatment method according to the present invention is contemplated to comprise the initial step of assessing a patient's skin wound site 100 by a trained or skilled health care provider. In this regard, it is contemplated that the involvement of a trained health care provider is an important feature to the practiced methodology insofar as the wound site 100 must be addressed in a competent manner. The trained or skilled health care provider will select a suturing technique based upon the assessed skin wound site 100 and in this regard will propose at least one, but possibly a number of suturing techniques for suture-closing the wound site 100.

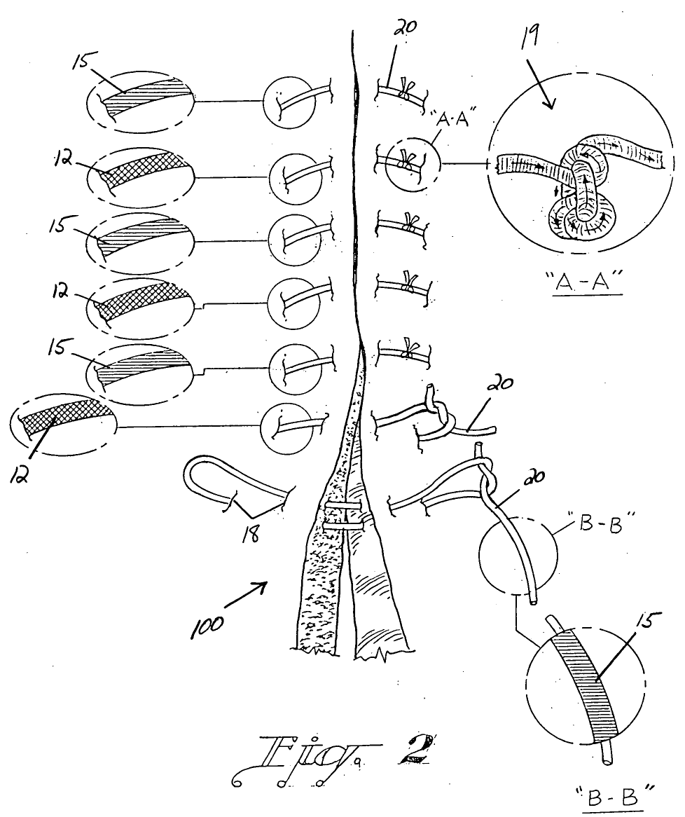

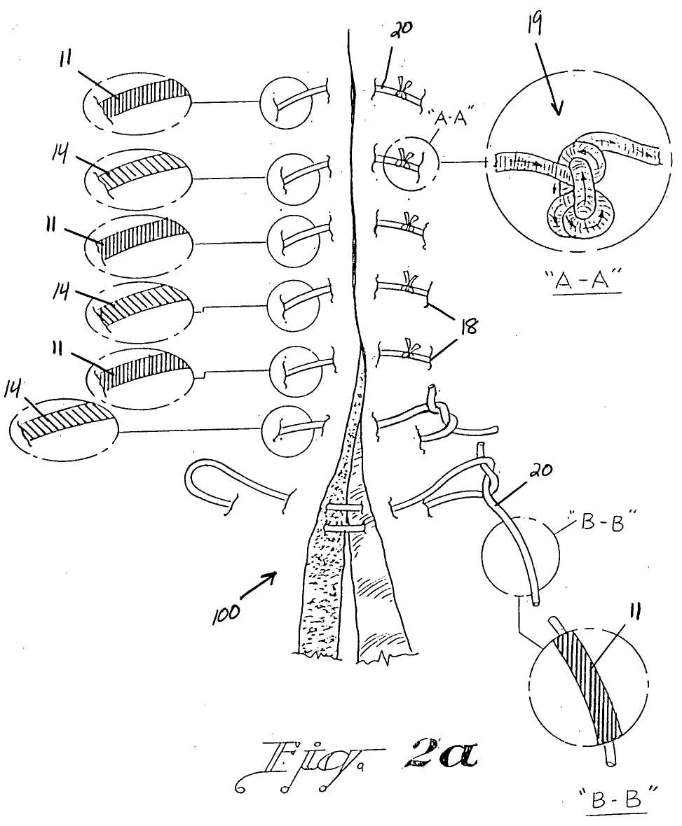

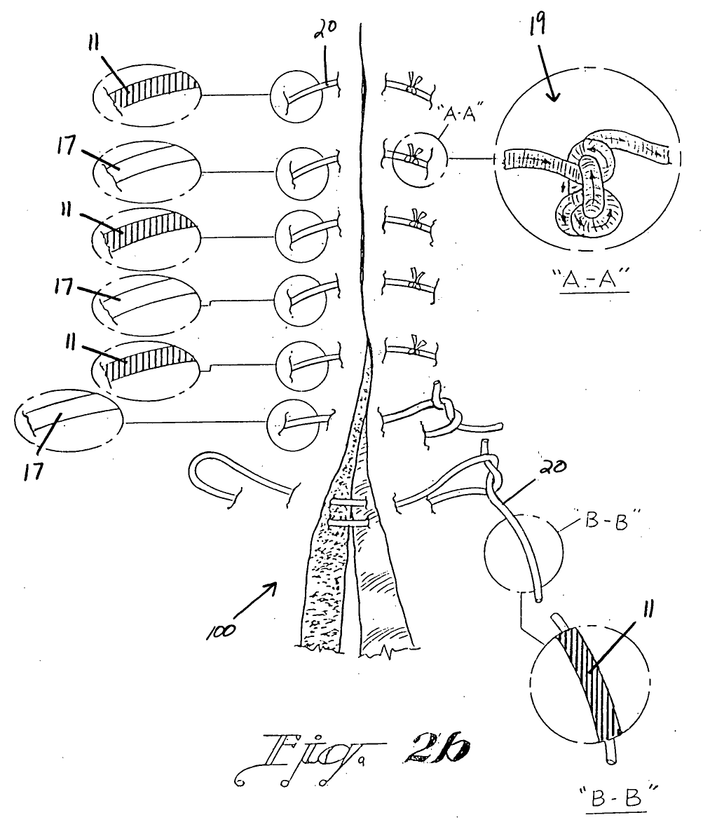

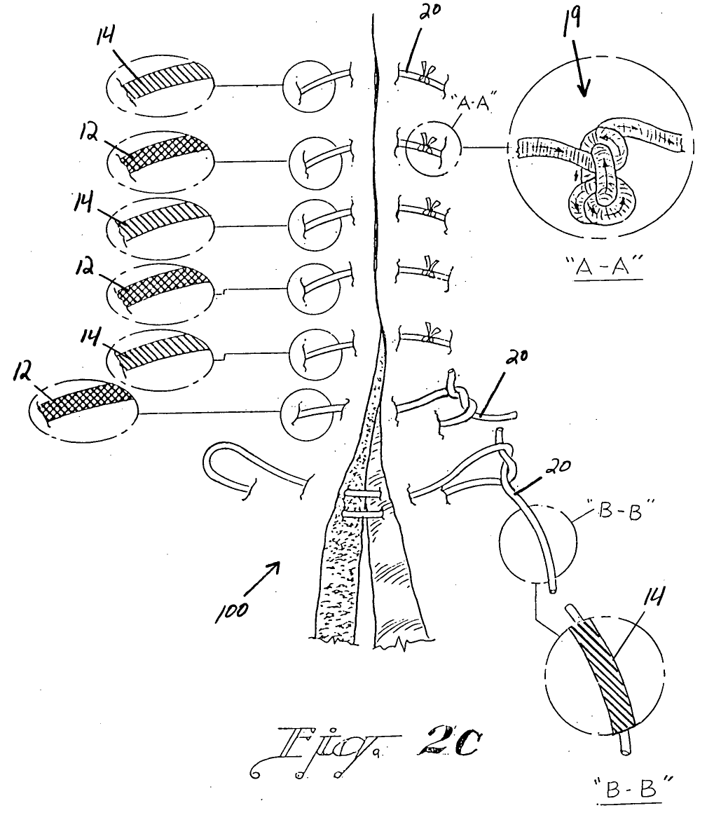

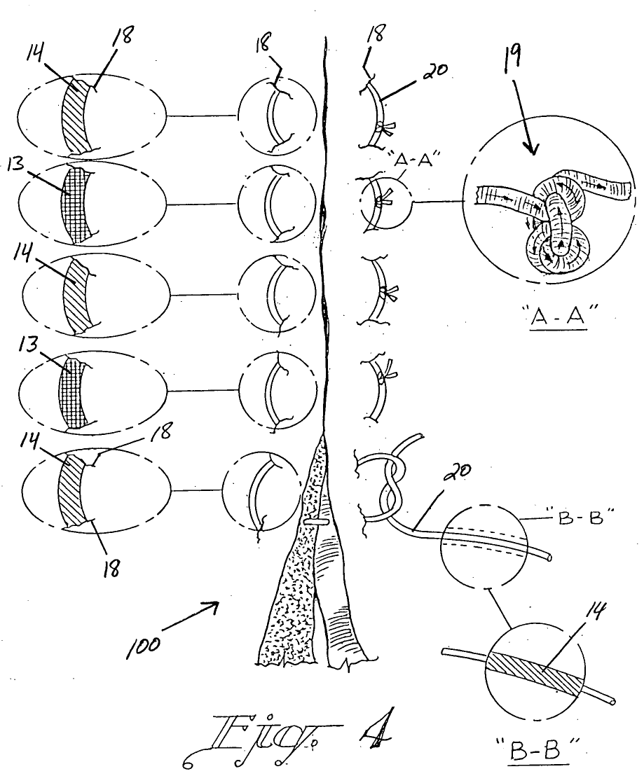

In this regard, it will be noted that FIG. 1 depicts a simple interrupted suture schematic; FIG. 2 shows a vertical mattress suture schematic; FIG. 3 shows a continuous, running, or over-and-over suture schematic; and FIG. 4 shows a horizontal mattress suture schematic. The illustrated suturing techniques are exemplary and not intended to be limiting. The chosen suturing technique selected by the health care provider will depend upon his or her training and practice sense for any given wound scenario.

Notably, proper suturing technique is essential to ensure good results in dermatologic surgery. The postoperative appearance of a beautifully designed closure or flap can be compromised if an incorrect suture technique is chosen or if the execution is poor. Conversely, meticulous suturing technique cannot fully compensate for improper surgical technique. Poor incision placement with respect to relaxed skin tension lines, excessive removal of tissue, or inadequate undermining may limit the surgeon's options in wound closure and suture placement. Gentle handling of the tissue is also important to optimize wound healing. The choice of suture technique depends on the type and anatomic location of the wound, the thickness of the skin, the degree of tension, and the desired cosmetic result. The proper placement of sutures enhances the precise approximation of the wound edges, which helps minimize and redistribute skin tension.

The most commonly used and versatile suture in cutaneous surgery is the simple interrupted suture as generally depicted in FIG. 1. This suture is placed by inserting the needle perpendicular to the epidermis, traversing the epidermis and the full thickness of the dermis, and exiting perpendicular to the epidermis on the opposite side of the wound. The 2 sides of the stitch should be symmetrically placed in terms of depth and width.

Compared with running sutures (as generally depicted in FIGS. 3 and 3(a)), interrupted sutures are typically easier to place, have greater tensile strength, and have less potential for causing wound edema and impaired cutaneous circulation. Interrupted sutures also allow the health care provider to make adjustments as needed to properly align wound edges as the wound is sutured. Disadvantages of interrupted sutures include the length of time required for their placement and the greater risk of crosshatched marks (i.e., train tracks) across the suture line. The risk of crosshatching can be minimized by removing sutures early to prevent the development of suture tracks.

The simple running suture or continuous over-and-over suture is generally illustrated in FIGS. 3 and 3(a). This type of suture technique employs an uninterrupted series of simple interrupted sutures. The suture is started by placing a simple interrupted stitch, which is tied but not cut. A series of simple sutures are placed in succession without tying or cutting the suture material after each pass. Sutures should be evenly spaced, and tension should be evenly distributed along the suture line. The line of stitches is completed by tying a knot (as generically depicted at 19) after the last pass at the end of the suture line. The knot is tied between the tail end of the suture material where it exits the wound and the loop of the last suture placed.

Running sutures are useful for long wounds in which wound tension has been minimized with properly placed deep sutures and in which approximation of the wound edges is good. This type of suture may also be used to secure a split- or full-thickness skin graft. Theoretically, less scarring occurs with running sutures compared with interrupted sutures because fewer knots are made with simple running sutures; however, the number of needle insertions remains the same.

Advantages of the simple running suture include quicker placement and more rapid re-approximation of wound edges, compared with simple interrupted sutures. Disadvantages include possible crosshatching, the risk of dehiscence if the suture material ruptures, difficulty in making fine adjustments along the suture line, and puckering of the suture line when the stitches are placed in thin skin.

The vertical mattress suture (as generally depicted in FIGS. 2-2(c)) is a variation of the simple interrupted suture. It consists of a simple interrupted stitch placed wide and deep into the wound edge and a second more superficial interrupted stitch placed closer to the wound edge and in the opposite direction. The width of the stitch should be increased in proportion to the amount of tension on the wound. That is, the higher the tension, the wider the stitch.

A vertical mattress suture is especially useful in maximizing wound eversion, reducing dead space, and minimizing tension across the wound. One of the disadvantages of this suture is crosshatching. The risk of crosshatching is greater because of increased tension across the wound and the 4 entry and exit points of the stitch in the skin. Placing each stitch precisely and taking symmetric bites is especially important with this suture.

The horizontal mattress suture (as generally depicted in FIG. 4) is placed by entering the skin 5 mm to 1 cm from the wound edge. The suture is passed deep in the dermis to the opposite side of the suture line and exits the skin equidistant from the wound edge (in effect, a deep simple interrupted stitch). The needle reenters the skin on the same side of the suture line 5 mm to 1 cm lateral of the exit point. The stitch is passed deep to the opposite side of the wound where it exits the skin and the knot is tied.

The horizontal mattress suture is useful for wounds under high tension because it provides strength and wound eversion. This suture may also be used as a stay stitch to temporarily approximate wound edges, allowing placement of simple interrupted or subcuticular stitches. The temporary stitches are removed after the tension is evenly distributed across the wound.

Horizontal mattress sutures may be left in place for a few days if wound tension persists after placement of the remaining stitches. In areas of extremely high tension at risk for dehiscence, horizontal mattress sutures may be left in place even after removal of the superficial skin sutures. However, they have a high risk of producing suture marks if left in place for longer than 7 days. Horizontal mattress sutures may be placed prior to a proposed excision as a skin expansion technique to reduce tension. Improved eversion may be achieved with this stitch in wounds without significant tension by using small bites and a fine suture.

In addition to the risk of suture marks, horizontal sutures have a high risk of tissue strangulation and wound edge necrosis if tied too tightly. Taking generous bites, using bolsters, and cinching the suture only as tightly as necessary to approximate the wound edges may decrease the risk, as does removing the sutures as early as possible. Placing sutures at a greater distance from the wound edge facilitates their removal.

It will thus be seen that suture technique and selection may be greatly benefited by input from a trained health care provider. Further, however, it will be seen that each technique provides a different visual presentation in superficial adjacency to the patient's skin at the wound site 100. For this reason it is contemplated that the present methodology may involve the step of providing a patient with a visual depiction of exemplary suture layout schema. Exemplary suture layout schemas are believed to be represented in FIGS. 1-4, inclusive. Notably, the suture layout schemas are reflective of the selected suturing technique.

Once the wound site has been assessed and a proper suturing technique decided upon, the patient may be provided with a suture color palette or selection of suture colors. In this last regard, it is contemplated that the suture color palette may well comprise any number of suture colors, including colors of varying electromagnetic wavelength ranging from about 400 nanometers (nm) to about 700 nanometers (nm), which wavelength range is roughly reflective of the visible color spectrum.

In this regard, FIG. 1 depicts a representative array or pattern of colored interrupted sutures, whereby red (700 nm) is referenced at 11; orange (600 nm) is referenced at 12; yellow (580 nm) is referenced at 13; green (530 nm) is referenced at 14; blue (480 nm) is referenced at 15; and purple or violet (400 nm) is referenced at 16. The resulting color patter red 11 (R), orange 12 (O), yellow 13 (Y), green 14 (G), blue 15 (B), and violet 16 (V), may be represented by ROY G BV as reflective of the famous mnemonic ROY G. BIV for the sequence of hues in the visible spectrum, in rainbows, and in order from longest to shortest electromagnetic wavelength. Other colors may include white 17, black, any shade of gray, and any variation thereof.

The patient may thus select any color from the suture color palette or selection of suture colors and the skin wound site may then be sutured with at least one suture, each suture comprising suture coloration reflective of the selected suture color(s). For example, the patient may select any number of colors such as red 11, orange 12, yellow, 13, green 14, blue 15, and violet 16 and pattern or arrange these colors to represent the visible spectrum or rainbow as generally depicted in FIG. 1.

Alternatively, the patient may select a color scheme (color and pattern) comprising alternating blue (15)—orange (12) colors to represent, for example, a favorite team color scheme as generally depicted in FIG. 2 or alternating green (14)—yellow (13) as generally depicted in FIG. 4. Further, the patient may select a color scheme comprising alternating red (11)—green (14) as generally depicted in FIG. 2(a); or red (11)—white (17) as generally depicted in FIG. 2(b); or green (14)—orange (12) to represent a holiday-themed color scheme (e.g. Christmas or Thanksgiving). FIG. 3(a) depicts a red (11)—white (17)—blue (15) color scheme generally representative of a 4^{th} of July or Independence Day-type holiday theme.

In this last regard, it should be noted that the length of the suture 20 or suture structure may comprise various colors. In other words, each suture 20 may be multi-colored along its length. FIG. 3(a), for example, shows a continuous length of suture 20 having multiple colors (i.e. red (11), white (17), and blue (15)) along its length. It is contemplated that the demarcations between adjacent colors along the length of the suture 20 may well be spaced to help guide the health care provider for suture—tissue bites during the suturing process.

In other words, differing suturing techniques and wound sites may require different suture-to-tissue bites. A selected suture structure 20, for example, may provide color-demarcated or color-banded sutures for guiding the suture installer during the suturing process. Exposed lengths of adjacent banded colors may well represent the proper suture-to-tissue bite widths. The lines of color demarcation may thus be roughly represented at minor incisions 18. The banded suture coloration may thus be said to provide certain suture-to-tissue bite width guide means for approximating proper suture-to-tissue bite width.

While the foregoing specifications and attached drawings are set forth in some detail, the specific procedures and supporting embodiments described and illustrated are to be considered as exemplifications of the principles of the invention and are not intended to limit the invention(s) to the specific methods and/or embodiments illustrated or described. For example, it is contemplated that the present invention essentially discloses a skin wound treatment method or suturing method.

The skin wound treatment method or suturing method according to the present method is essentially set forth for externally adorning a skin wound site or suture site. The skin wound treatment method may be said to comprise the steps of: assessing a patient's skin wound site, which site is assessed by a medical or health care provider. A suturing technique is then selected as based upon the assessed skin wound site. When a suturing technique is decided upon, the patient may be provided with visual depiction(s) of exemplary suture layout schema, which schema are reflective of the selected suturing technique. The patient may thus obtain a visual depiction of how his or her suture, when completed with structurally appear.

When the patient has an understanding for how the suture 20 will appear adjacent the wound site, he or she will have a better opportunity to select any number of suture colors as provided the patient via a suture color palette or selection of suture colors. Further, the patient then has an opportunity to arrange or pattern the colors according to his or her selections and the preferred suture technique. The skin wound site may then be sutured with one or more lengths of suture 20, each of which comprises suture coloration reflective of the patient-selected suture color(s).

Stated another way, the suturing method according to the present invention may be said to essentially comprise the steps of: providing a patient with a selection of suture colors; inviting the patient to select at least one suture color from the suture color palette; selecting at least one suture color from the selection of suture colors; and suturing a suture site with a suture, the suture comprising the selected suture color. The method may be said to comprise the additional steps of assessing the suture site; selecting a suturing technique based on the assessed suture site; and providing a visual depiction of exemplary suture layout schema to the patient, the exemplary suture layout schema being reflective of the selected suturing technique.

The selection of suture colors may comprise any color, including colors of varying electromagnetic wavelengths ranging from about 400 nm to about 700 nm. The sutures may further comprise a suture length, which length may comprise colors of varying electromagnetic wavelengths along the entire visible spectrum. The colors may be spaced along the length of the suture length to serve as bite length guide way demarcations for the suture(s) 20.

The suturing method may be supported by a suture kit comprising any number of suture colors and styles according to the presenting methodology and supporting embodiment(s), along with suture needles of any type and style. Preferably, however, it is contemplated that the kit may include or comprise sutures either permanently or temporarily swaged to needles.

From the foregoing, it will be observed that numerous variations and modifications of the disclosed methodology and underlying suture structure 20 may be effected without departing from the spirit and scope of the invention. Thus, it is to be understood that no limitation with respect to the specific structure(s) and/or methodology illustrated herein is intended or should be inferred. It is, of course, intended to cover by the appended claims all such modifications as fall within the scope of the claims. ** Portions of this detailed description were adapted from:

(1) Suturing Techniques, Julian MacKay-Wiggan, MD & Désirée Ratner, MD, http://emedicine.medscape.com/article/1128240; and(2) http://en.wikipedia.org/wiki/Surgical_suture

I claim:

1. A skin wound treatment method, the skin wound treatment method for externally adorning a skin wound site, the skin wound treatment method comprising the steps of:
assessing a patient's skin wound site, the patient's skin wound site being assessed by a health care provider;
selecting a suturing technique based on the assessed skin wound site, the suturing technique being selected by the health care provider;
providing a patient with a visual depiction of exemplary suture layout schema, the suture layout schema being reflective of the selected suturing technique;
providing the patient with a suture color palette, the suture color palette comprising a plurality of suture colors;
selecting at least one suture color from the suture color palette, each suture color being selected by the patient; and
suturing the skin wound site with at least one suture, each suture comprising suture coloration, the suture coloration being reflective of the selected suture color(s).

2. The method of claim 1 wherein the suture color palette comprises colors of varying electromagnetic wavelengths, the varying electromagnetic wavelengths ranging from about 400 nm to about 700 nm.

3. The method of claim 1 wherein each suture comprises a suture length, each suture length comprising colors of varying electromagnetic wavelengths, the electromagnetic wavelengths ranging from about 400 nm to about 700 nm.

4. The method of claim 1 comprising the steps of:
selecting at least two suture colors from the suture color palette, each suture color being selected by the patient; and
suturing the skin wound site with a plurality of sutures, each suture comprising select suture coloration, the selected suture coloration being reflective of the selected suture colors.

5. The method of claim 4 wherein the selected suture colors are patterned, the selected suture colors being patterned according to the patient's instructions before suturing the skin wound site with the plurality of sutures.

6. The method of claim 1 comprising the steps of:
selecting at least three suture colors from the suture color palette, each suture colors being selected by the patient; and
suturing the skin wound site with a plurality of sutures, each suture comprising select suture coloration, the selected suture coloration being reflective of the selected suture colors.

7. The method of claim 6 wherein the selected suture colors are patterned, the selected suture colors being patterned according to the patient's instructions before suturing the suture site with the plurality of sutures.

8. A suturing method, the suturing method for adorning a suture site, the suturing method comprising the steps of:
providing a patient with a selection of suture colors;
selecting at least one suture color from the selection of suture colors; and
suturing a suture site with a suture, the suture comprising the selected suture color.

9. The method of claim 8 comprising the steps of:
assessing the suture site;
selecting a suturing technique based on the assessed suture site; and
providing a visual depiction of exemplary suture layout schema to the patient, the exemplary suture layout schema being reflective of the selected suturing technique.

10. The method of claim 8 wherein the selection of suture colors comprises colors of varying electromagnetic wavelengths, the varying electromagnetic wavelengths ranging from about 400 nm to about 700 nm.

11. The method of claim 8 wherein each suture comprises a suture length, the suture length comprising colors of varying electromagnetic wavelengths, the varying electromagnetic wavelengths ranging from about 400 nm to about 700 nm.

12. The method of claim 8 comprising the steps of:
selecting at least two suture colors from the selection of suture colors; and
suturing the suture site with sutures comprising the selected suture colors.

13. The method of claim 12 wherein the selected suture colors are patterned according to the patient's instructions before suturing the suture site.

14. The method of claim 8 comprising the steps of:
selecting at least three suture colors from the selection of suture colors; and
suturing the suture site with sutures comprising the selected suture colors.

15. The method of claim 14 wherein the selected suture colors are patterned according to the patient's instructions before suturing the suture site.

16. A suture kit, the suture kit for enabling a suture provider to adorn a suture site with patient-selected colored sutures, the suture kit comprising:
a plurality of sutures, each suture comprising suture coloration, the suture coloration comprising varying electromagnetic wavelengths, the varying electromagnetic wavelengths ranging from about 400 nm to about 700 nm.

17. The kit of claim 16 wherein the sutures each comprise a suture length, the suture lengths comprising banded suture coloration of varying electromagnetic wavelengths, the varying electromagnetic wavelengths ranging from about 400 nm to about 700 nm.

18. The kit of claim 17 wherein the banded suture coloration provides suture-to-tissue bite width guide means for approximating proper suture-to-tissue bite width.

19. The kit of claim 18 comprising at least one surgical needle.

20. The kit of claim 19 wherein the surgical needle is swaged to a select suture.
